(19) United States
(12) Patent Application Publication
(10) Pub. No.: US 2004/0171931 A1
(43) Pub. Date: Sep. 2, 2004

(54) Method to produce a volume data set

(76) Inventors: Karl Barth, Hoechstadt (DE); Gerd Wessels, Effeltrich (DE)

Correspondence Address: SCHIFF HARDIN, LLP, PATENT DEPARTMENT, 6600 SEARS TOWER, CHICAGO, IL, 60606-6473, US

(21) Appl. No.: 10/721,931

(22) Filed: Nov. 25, 2003

(30) Foreign Application Priority Data
Nov. 25, 2002 (DE) 10254943.5

(51) Int. Cl. A61B 5/05; G06F 17/00

(52) U.S. Cl. 600/425; 128/922; 382/128

(57) ABSTRACT

In a method to produce a volume data set, the imaged surface of a subject imaged in a first volume data set is segmented. The first volume data set then is transformed into a second volume data set, such that the segmented imaged surface is transformed into a plane. From the second volume data set, a third volume data set is produced by filtering the second volume data set such that structures not of interest of the first subject, imaged in the second volume data set, are filtered out based on features associated with structures not of interest and based on the expected removals from the surface of the structures not of interest, and structures of interest of the first subject, imaged in the second volume data set, remain based on features associated with structures of interest, and based on the expected removals of the structures not of interest from the surface.

### BACKGROUND OF THE INVENTION

1. Field of the Invention

The present invention relates to a method for producing an image from a three-dimensional image of a subject.

2. Description of the Prior Art

Images that are picked up with modern imaging medical devices have a relatively high resolution in all directions, and therefore amplified 3D projections (volume datasets) are generated with them. Imaging medical devices include ultrasound devices, computed tomography devices, magnetic resonance devices or X-ray devices or PET scanners, for example. Computed tomography or X-ray devices can also frequently be utilized, because the radiation load to which a living being is exposed during an examination with one of these devices has decreased. Volume datasets contain a larger amount of data than image datasets of conventional two-dimensional images, which is why an evaluation of volume datasets is relatively time-consuming. The actual pick-up of the volume datasets currently takes approximately half a minute, but it frequently takes half an hour or more to search through and edit the volume dataset. Methods for automatic recognition and editing are needed and desirable.

Until the year 2000, it was customary practice in computed tomography (CT) to reach a diagnosis based almost exclusively on axial slice stacks (slice images) or at least to focus findings predominantly on the slice images. Since 1995, due to the power of computers, 3D representations on diagnostic consoles have been widespread; initially they had a scientific or ancillary importance. Essentially four basic methods of 3D visualization were developed in order to facilitate diagnosis by a physician:

1. Multiplanar reformatting (MPR): This is no more than a reconfiguration of the volume dataset in a different orientation from the original horizontal slices. It basically breaks down into orthogonal MPR (three MPRs, respectively perpendicular to a coordinate axis), free MPR (oblique slices; derivative=interpolated) and curved MPR (slice representation parallel to an arbitrary path through the image of the body of the living being and e.g. perpendicular to the MPR in which the path was drawn).

2. Shaded Surface Display (SSD): segmenting the volume dataset and representing the surface of the subject that is being cut, usually strongly influenced by orienting at the CT values and manual help editing.

3. Maximal Intensity Projection (MIP): representation of the highest intensity along each ray. In what is known as thin MIP, only a sub-volume is represented.

4. Volume rendering (VR): encompasses modeling of the attenuation of the ray that penetrates the subject in a similar manner as an X-ray. The entire depth of the imaged body (partly translucent) is captured; however, details of small and above all thin-sliced subjects are lost. The representation is influenced manually by the setting of what are known as transfer functions (color lookup tables).

Although volume rendering (VR) offers relatively good results, images processed with volume rendering can exhibit a limited transparency. The transparency can be ensured only with restrictions over the entire range (area), by a change of the transfer function. It may occur that the sole transparent representation of specific diseased structures, such as, for example, a diseased organ, is possible limited only by a setting of the transfer function. Editing functions then can be used in the field of volume rendering in order to segment out (at least with manual support) and then to separately represent the desired imaged structures. The desired imaged structures often must be completely manually segmented. If the structures are imaged with a volume data set that exists in the form of a number of successive computed tomographic slice images, the contours of the imaged structures are thus manually circumscribed slice by slice, such that only the voxels in the closed contours are shown.

### SUMMARY OF THE INVENTION

An object of the present invention is to provide a method to produce a volume data set from a volume data set representing an imaged subject, in which structures of interest of the imaged subject are shown in an improved manner.

This object of the invention is achieved in accordance with the invention by a method to produce a volume data set, including the steps of segmenting the imaged surface of a subject imaged in a first volume data set, transforming the first volume data set into a second volume data set, such that the segmented imaged surface is transformed into a plane, and producing a third volume data set, by filtering the second volume data set such that structures not of interest of the subject, imaged in the second volume data set, are filtered out based on features associated in general with structures not of interest and based on the expected removals from the surface of the structures not of interest, and structures of interest of the subject, imaged in the second volume data set, remain based on features associated with structures of interest in general, and based on the expected removals of the structures not of interest from the surface.

In the first volume data set, at least one part of the subject (that, for example, is a living organism) is imaged. If, with an image associated with the first volume data set, structures lying inside the subject should be examined, the images of other structures of the subject disposed closer to the surface can shadow the deeper-situated imaged structures, or cover their representation. By means of the inventive method, the imaged structures disposed closer to the surface of the subject should be filtered out as much as possible, without removing imaged structures that should be examined (structures of interest) and that are arranged deeper inside the subject.

In accordance with the invention, the surface of the imaged subject represented in the first volume data set is determined (segmented out), preferably automatically. In the case of a living organism as the first subject, this normally curved surface is transformed into the plane, as if the imaged subject were unrolled. An analogy is the projection of the earth's surface on maps. In particular when the imaged subject is the imaged torso of the living organism that resembles a columnar shape with an approximately elliptical base, the imaged surface (meaning the imaged body surface of the living organism) can be unrolled into a planar surface. The second volume data set thus is obtained.

The structures to be filtered out are subsequently filtered out of the second volume data set with a suitable filter or a suitable set of filters. Thus for each type or class of structures to be filtered out (for example, skin, fat, ribs, bones or muscles), a filter is determined. The respective filters are developed according to the features associated with the structures to be filtered out. In an embodiment of the invention, density-oriented, texture-oriented, edge-sensitive and/or morphological filtering associated with the structures not of interest is used. The individual filter responses can finally be suitably added together in a feature matrix.

Furthermore, structures deeper inside the first subject should be examined. Therefore, for the filtering, the expected removal of the structures to be filtered out from the surface of the first subject is considered for the filtering out of the non-structures of interest.

If the subject is a living organism and the first data set represents a part of the body, for example the abdomen region of a person, and the structures of interest are the spinal column and the inner organs of the person, the structures not of interest are skin, ribs and fatty tissue. Thus respective filters are determined for filtering out the imaged skin, the imaged ribs, and the imaged fatty tissue, with each filter recognizing the image of the corresponding structure that the filter should filter out.

The filter that should filter out the imaged ribs is, for example, a filter recognizes the imaged bones. However, so that the imaged spinal column (which is a part of the structures of interest) is not filtered out, filters associated with the imaged bones should operate only for a region of the imaged body that corresponds to a specific depth from the body surface in the inside of the body. This removal corresponds to the expected removal of ribs from a body layer inside the person. It is thus ensured that no part of the imaged spinal column is filtered out by mistake.

In contrast to this, the filter associated with the skin should operate only for a region of the imaged body that corresponds to the thickness of the skin of the person. The filter associated with the fatty tissue should likewise operate only for a region of the imaged body that corresponds to a predetermined removal of a body layer in the inside of the body. This removal is selected such that no region of the imaged body is considered in which structures of interest are imaged.

For the individual filters, among other things the topology of the inside of the body of the living organism and the expected region of the structures of interest is are considered. The structures of interest are structures arranged deeper inside the body of the living organism, such as, for example, inner organs. Structures near to the surface should therefore be filtered out. For taking into account the expected removal of structures not of interest in the body of the living organism, spatial probabilities (higher weighting near the surface) of the structures not of interest therefore can be used.

The features associated with the structures not of interest are, for example, determined by an edge definition in the direction of the imaged body surface inside the imaged body. Thus, for example, a relatively significantly formed intensity decrease results for imaged lungs or at surface-proximate gas bubbles in the abdominal area.

The subject may also be a technical (inanimate) subject, in the image of which, for example, an imaged coating or imaged insulation of the technical subject should be filtered out as structures not of interest.

According to an embodiment of the invention, if the first volume data set contains at least one imaged additional (second) subject that is disposed outside of the first subject, the imaged second subject can be filtered out of the second volume data set with the non-interesting imaged structures. The second subject may be, for example, a table on which the first subject lies during the acquisition of the first volume data set, clothing of the living organism, or instruments arranged at a living organism.

In a preferred embodiment of the invention, the imaged surface can be segmented when the first volume data set exists in the form of a number of successive computed tomographic slice images or is considered as a slice stack, the image data of each slice image being described with Cartesian coordinates, and wherein the following method steps are implemented for the segmentation of the imaged body surface.

A coordinate transformation for each slice image to polar coordinates is implemented with regard to a straight line that proceeds through the imaged subject and that is aligned substantially at a right angle to the individual slice images. Contours are determined that are imaged in each transformed slice image and are associated with the imaged surface. The image points of the determined contours are transformed back into the coordinate system associated with the first volume data set. Image points are re-extracted along the contours for the representation of the surface of the imaged subject transformed in the plane.

In an embodiment of the invention, a fourth volume data set is additionally produced in which the image points of the third volume data set are transformed back into the coordinate system associated with the first volume data set. The fourth volume data set thus contains the structures of interest imaged in the first volume data set.

The fourth volume data set can be used in order to represent, by means of volume rendering (VR), an image associated with the fourth volume data set. Non-filtered-out, structures not of interest thus have a minimally negative influence, because for the actual structures to be represented the transfer function is the substantial level control, and the character of the mixing technique is very insensitive when “in advance”, for example, structures not of interest of a few millimeters are located at each ray. The structures not of interest still present in the fourth volume data set are, with high probability, barely visible, since relatively high-contrast structures not of interest are normally filtered out.

### DESCRIPTION OF THE DRAWINGS

FIG. 1 is a computed tomography apparatus operable in accordance with the invention.

FIG. 2 shows a volume data set of the abdominal area of a patient, as a volume data set containing a number of slice images.

FIG. 3 is a slice image of the volume data set shown in FIG. 2.

FIG. 4 shows image information of the slice image shown in FIG. 3, transformed to polar coordinates,

FIG. 5 shows a further volume data set, in which the imaged body surface of the volume data set shown in FIG. 2 is transformed into a plane.

FIG. 6 shows a further volume data set that, to the extent possible, contains only imaged structures of interest of the volume data set shown in FIG. 5.

FIG. 7 is a representation of the volume data set shown in FIG. 2, processed by means of volume rendering.

FIG. 8 is a representation of the volume data set shown in FIG. 2, processed by means of volume rendering, in which imaged structures not of interest of the volume data set shown in FIG. 2 are filtered out.

### DESCRIPTION OF THE PREFERRED EMBODIMENTS

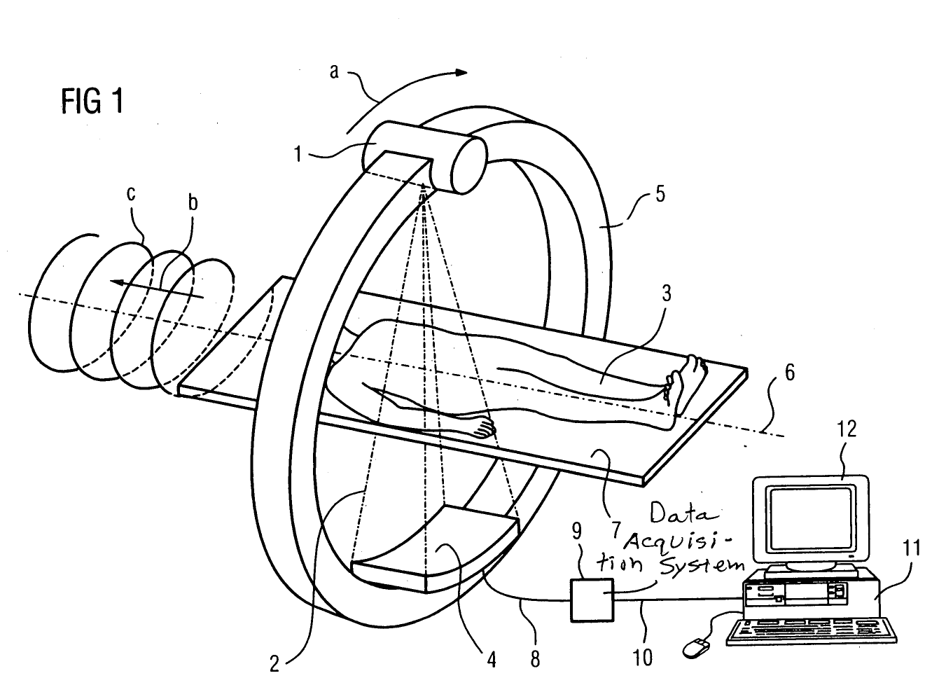

FIG. 1 is a schematic representation of a computed tomography apparatus with an X-ray source 1 which emits a pyramidal X-ray beam 2 the peripheral rays of which are represented as dotted lines in FIG. 1, which passes through an examination subject, for instance a patient 3, and strikes a radiation detector 4. This X-ray source 1 and the X-ray detector 4 are disposed facing one another on opposite sides of an annular gantry 5. The gantry 5 is supported by a bearing device that is not shown in FIG. 1, such that it pivots relative to a system axis 6 that extends through the midpoint of the annular gantry 5 (arrow a).

In the exemplary embodiment, the patient 3 lies on a table 7 that is transparent to X-ray, which is supported by means of a bearing device that is not shown in FIG. 1 in such a way that it can be displaced along the system axis 6 (arrow b).

The X-ray source 1 and X-ray detector 4 form a measuring system which is rotatable relative to the system axis 6 and displaceable along the system axis 6 relative to the patient 3, so that the patient can be irradiated at different projection angles and different positions relative to the system axis 6. From the generated output signals of the radiation detector 4, a data acquisition system 9 forms measurement values, which are fed to a computer 11, which computes, by methods known to those skilled in the art, an image of the patient 3 that can be reproduced on a monitor 12 that is connected to the computer 11. In the exemplary embodiment, the data acquisition system 9 is connected to the radiation detector 4 by an electrical line 8, which terminate in a wiper ring system, or a wireless transmission path, to obtain signals from the radiation detector 4, and is connected to the computer 11 by an electrical line 10.

The computed tomography apparatus shown in FIG. 1 can be utilized for sequential scanning and spiral scanning.

In sequential scanning, the patient 3 is scanned slice by slice. The X-ray source 1 and the X-ray detector 4 are rotated around the patient 3 relative to the system axis 6, and the measuring system, which includes the X-ray source 1 and the X-ray detector 4, captures a number of projections in order to scan a two-dimensional slice of the patient 3. From the measurement values so acquired, a slice image representing the scanned slice is reconstructed. Between the scanning of consecutive slices, the patient 3 is moved along the system axis 6. This process is repeated until all relevant slices are picked up.

During a spiral scan, the measuring system formed by the X-ray source 1 and the X-ray detector 4 rotates relative to the system axis 6, and the table 7 moves continuously in the direction of arrow b; that is, the measuring system comprising the X-ray source 1 and the X-ray detector 4 continuously moves on a spiral path c relative to the patient 3 until the region of interest of the patient 3 is completely covered. A volume dataset is thereby generated, which is coded according to the customary DICOM standard in the present embodiment.

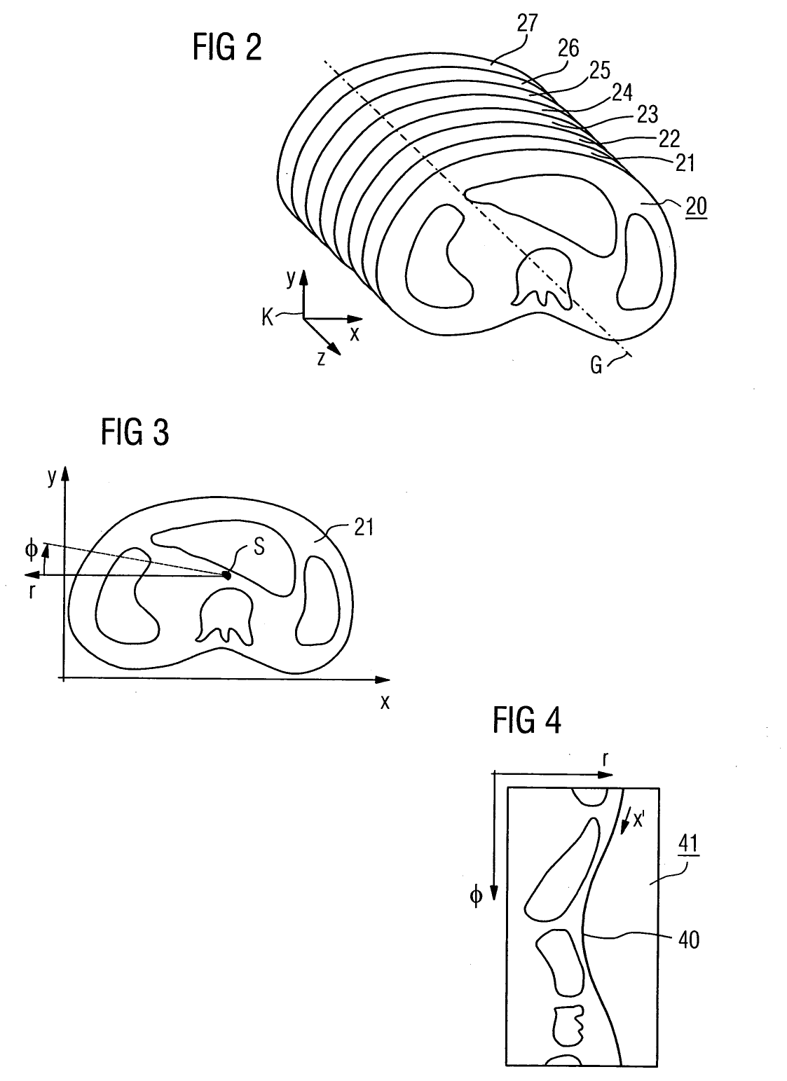

In the exemplary embodiment, a volume data set 20 of the abdominal area of the patient 3, formed by a number of successive slice images, is produced with the computed tomography apparatus shown in FIG. 1. In the exemplary embodiment, the volume data set 20 (that is schematically shown in FIG. 2) contains approximately 250 CT slices (slice images) of the matrix 512×512. In FIG. 2, seven slice images that are provided with the reference characters 21 through 27 are indicated for example.

In the exemplary embodiment, imaged structures near to the surface of the body that are imaged with (contained in) the volume data set 20 should be filtered out, such that, to the extent possible, only imaged inner organs and the imaged spinal column of the patient 3 are visible. For this, in the exemplary embodiment, a suitable computer program runs on the computer 11 that implements the steps specified below.

First, in a first pass to determine the imaged body surface, each slice image 21 through 27 of the volume data set 20 is transformed to polar coordinates with regard to a straight line G that proceeds through the three-dimensional image of the abdominal area of the patient 3. The straight line G is substantially aligned at right angles to the individual slice images 21 through 27. In the exemplary embodiment, the straight line G proceeds substantially through the center of the volume data set 20 and corresponds to the z-axis of the coordinate system K defining the volume data set.

In the exemplary embodiment, each slice image 21 through 27 (of which the slice image 21, as an example, is shown in FIG. 3) is described with Cartesian coordinates (x, y). Subsequently, the image information of each slice image 21 through 27 is radially rearranged, by transformation to polar coordinates (r, φ) with regard to the straight line G, or with regard to the respective slice points between the straight line G and the corresponding slice image. As an example, the slice point S between the straight line G and the slice image 21 is shown in FIG. 3. With the transformation to polar coordinates (r, φ), the image of the body surface of the patient 3 is also transformed and shown as a contour in each transformed axial slice (slice image). A contour 40 associated with the image of the body surface of the patient 3 shown, as an example, in FIG. 4 for the slice image 21 transformed according to polar coordinates (r, φ). The transformed slice image of the slice image 21 is provided with the reference character 41.

The result of the transformation to polar coordinates (r, φ) is a linearly plotted radial brightness profile. In this rectangular matrix (derived image matrix), filtering is now implemented which emphasizes the coritours associated with the body surface, such as the contour 40 shown in FIG. 4. The filter response of one or more employed filters replaces the brightness values in the derived image matrix. The search for the optimal path in this image matrix now ensues from top to bottom at the identical start/target point. In the exemplary embodiment, this ensues by means of dynamic optimization, as specified, for example, in R. Bellman, “Dynamic programming and stochastic control processes”, Information and Control, 1(3), pages 228-239, September 1958. The optimized path represents the radial vectors at the body surface image points. In a further step, a transformation of the contours 40 (transformed to polar coordinates) back into the original coordinates (x, y, z) of the volume data set 20 ensues, such that the entire contour ensemble specified by the individual contours of the slice images, and the corresponding image points of the original volume data set 20, are tested over all slice images 21 through 17 in the context of the individual contours. This contributes in particular to the suppression of errors (outliers) and to the reliability. In the exemplary embodiment, a re-segmentation in the individual slice images 21 through 27 is implemented at probable error locations with subsequent renewed testing of the 3D context. The image of the body surface of the patient 3 thus is segmented in the volume data set 20.

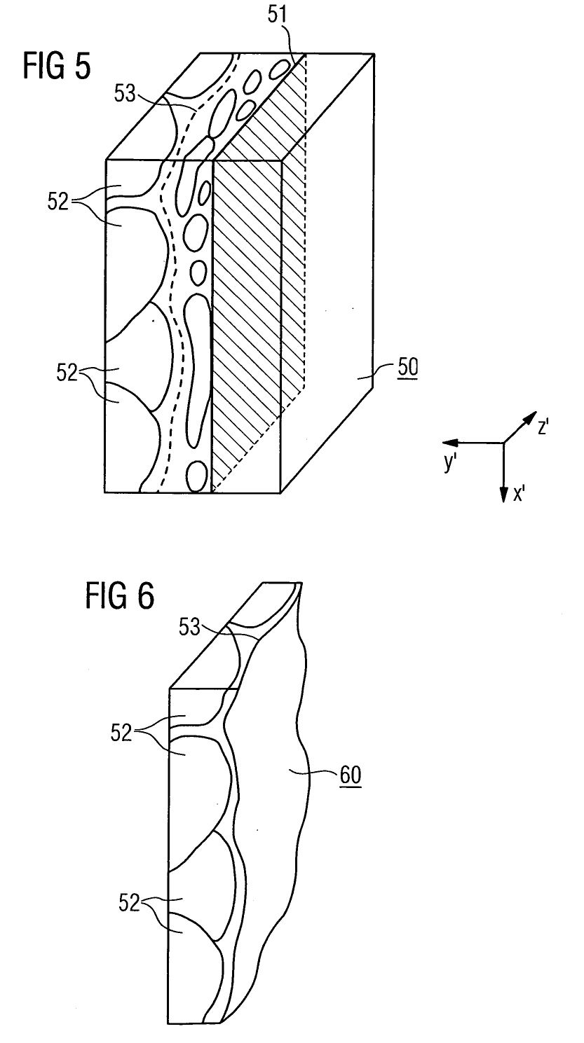

A re-extraction at right angles to the image of the segmented body surface subsequently ensues in the volume data set 20. While, in the transformation to polar coordinates (r, φ), brightness profiles were determined from the original data at right angles to all points of a circle (idealized surface contour) and plotted as a rectangular matrix, in the re-extraction profiles are acquired at right angles to each image point of the image of the segmented body surface (body surface contour). This re-extraction is newly plotted as a rectangular matrix. The volume data set 20 is thereby transformed such that the segmented image of the body surface of the patient 3 is transformed into a plane, and yields a volume data set 50 shown in FIG. 5 that has the structure of a voxel cube. The imaged body surface transformed into the plane is provided in FIG. 5 with the reference character 51, and is subsequently designated as a median (middle) plane 51. If the volume data set 20 contains horizontal slices (such as the slice images 21 through 27), each perpendicular line in the median plane 51 thus corresponds to the re-extracted volume data set 50 (right-angle voxel cube) of the image points of the image of the body surface in each of the slice images 21-27. The CT measurement values are located near the body surface inwards, left of this median plane 51, in the range of higher y′ coordinates. In that the form of a volume data set 50 comprising a voxel cube is ensured, the 3D context is ensured for a consistent segmentation. It is therefore well suited for the filtering to filter out structures not of interest imaged in the volume data set 50. In the exemplary embodiment, the imaged structures of interest are inner organs imaged in the volume data set 50 and the imaged spinal column.

In the exemplary embodiment, various filter operations are determined for different non-interesting imaged structures for the filtering out of the structures not of interest imaged in the volume data set 50. The filter operations take into account, among other things, specific features associated with the individual structures not of interest, and corresponding interval weightings to the body surface of the patient 3. For organs lying deeper, whose surfaces approach the body surface, feature filterings are applied which reduce the probability of the association with the non-interesting tissue layer. The distance to the body surface also considered here, with weighting. Also employed for this purpose are features that are determined by means of differentiating operators to recognize tissue contours such as, for example, rib surfaces inwards or organ surfaces from the inside out. All filter operations are subsequently merged in a probability matrix. The is done, depending on the operation, additively or multiplicatively with applicable scaling and suitable weightings.

When, as .in the present exemplary embodiment, the volume data set 50 is described with Cartesian coordinates (x′, y′, z′), and y′=const. is true for the image points of the imaged body surface 51 (meaning that the imaged body surface 51 is transformed into a plane), the x′-z′ planes of the volume data set are thus aligned parallel to the imaged body surface 51. The filtering of the volume data set 50, and thus the feature change of structures of interest to non-interesting (filtered-out) structures, therefore ensues substantially in the y′-direction.

In the combined probability matrix, as in the determination of the imaged body surface in the exemplary embodiment, the dynamic optimization is used in order to find an optimized path between the imaged body surface 51 and the imaged structures of interest, thus, in the case of the present exemplary embodiment, inner organs and the imaged spinal column 52. In the exemplary embodiment, this ensues in slice images associated with the volume data set 50 with a subsequent pass to ensure the context over the entire volume. The optimization in the individual slice images is actually one-dimensional, and therefore relatively efficient and fast. The production of the context ensues in the planar dimension; finally, however, 3D information is provided. Overall, a common 3D surface of the structures of interest present inside the imaged inside of the body of the patient 3 emerges. This 3D surface is provided with the reference character 53 in the volume data set 50, which exhibits the form of a voxel cube. The imaged area between the 3D surface 53 and the imaged body surface 51 contains the structures not of interest. This area is subsequently removed from the volume data set 50; it arises from a further volume data set 60 shown in FIG. 6.

In the exemplary embodiment, the image points of the volume data set 60 (containing, to the extent possible, only the imaged structures of interest 52) are transformed back into the original coordinate system K associated with the original volume data set 20 containing the slice images 21 through 27. Thus, from the volume data set 20 shown in FIG. 2, a volume data set ensues, from which, to the extent possible, many image points that are associated with non-interesting imaged structures in the volume data set 20 are filtered out, and that, to the extent possible, contains the image points that are associated with the structures of interest, thus the inner organs imaged in the volume data set 20 and the imaged spinal column. In this volume data set, another volume rendering of the imaged inner organs can subsequently be imaged. The result is an image 80 that, for example, is shown in FIG. 8.

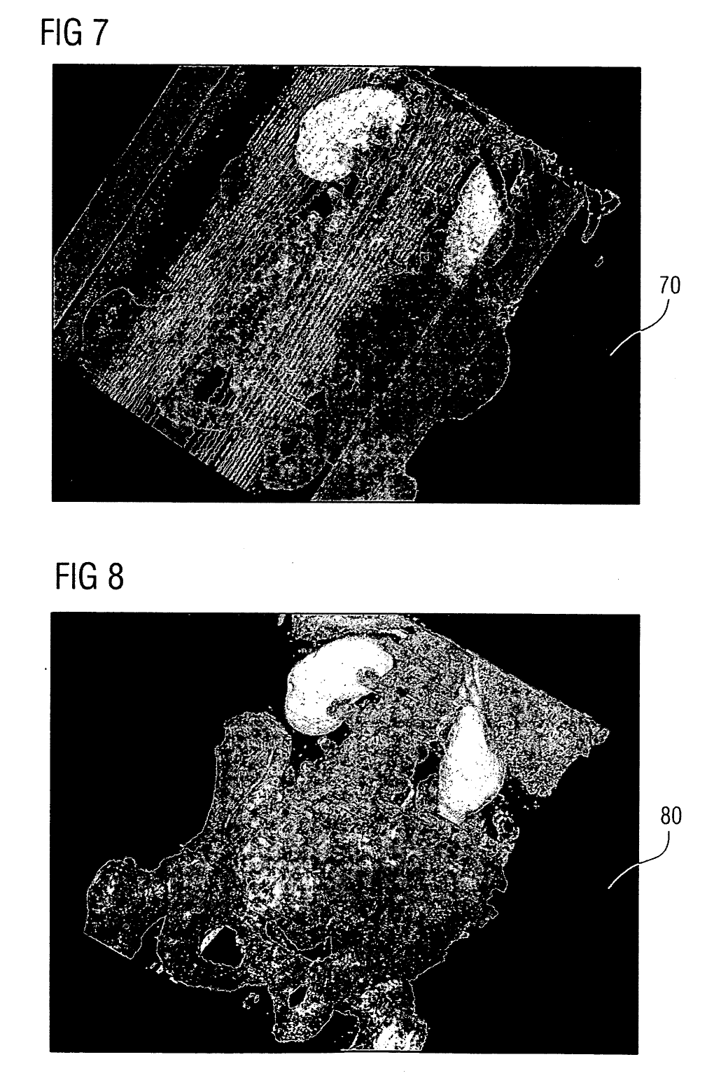

For comparison, FIG. 7 shows an image 70 that arose by implementing, for the original volume data set 20, a complete volume rendering without the processing disclosed herein.

A comparison of images 80 and 70 shows a number of the advantage of the inventive method:

Direct representation of the “leading inner” organs

Significantly clearer view of inner organs “in the second row”

Finer CT value resolution in the organs

Practice-compatible adjustment of various (color) sections in the transfer function, meaning also given relatively rough adjustment various colored subjects are clearly separated and well represented, which, without the specified preprocessing, would make the use of volume renders impossible in many environments.

In the exemplary embodiment, the volume data set is produced with a computer tomograph, and exists in the form of a number of successive computer-tomographic slice images. The volume data set alternatively can be produced with other imaging devices, such as in particular with a magnetic resonance device, an x-ray device, an ultrasound device, or a PET scanner. The volume data set also need not exist in the form of a number of successive computer-tomographic slice images.

In the exemplary embodiment, the volume data set 20 represents a part of the imaged body of the living organism 3. Alternatively, the volume data set can represent inanimate subjects, such as, for example, an image of the table 7 of the computed tomography apparatus, an image of the clothing of the patient 3, or an image of instruments on the patient 3 (not shown in FIG. 1).

The inventive method can also be used for imaged technical subjects. If, for example, the technical subject has a coating or an insulation, these can thus be removed as structures not of interest.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method to produce a volume data set, comprising the steps of:
segmenting an imaged surface of a subject imaged in a first volume data set;
transforming the first volume data set into a second volume data set, causing the segmented imaged surface to be transformed into a plane; and
producing a third volume data set by filtering the second volume data set such that structures not of interest of the subject, imaged in the second volume data set, are filtered out based on features associated in general with structures not of interest and based on expected removals from the surface of the structures not of interest, and such that structures of interest of the subject, imaged in the second volume data set (50), remain based on features associated with structures of interest, and based on the expected removals of the structures not of interest from the surface.

2. A method as claimed in claim 1, wherein the subject is a first subject and wherein at least one imaged second subject that is disposed outside of the first subject, and comprising filtering out the imaged second subject from the second volume data set with the non-interesting imaged structures.

3. A method as claimed in claim 1 comprising filtering the second volume data set by at least one of a density-oriented, texture-oriented, edge-sensitive and morphological filtering associated with at least one of the structures not of interest and the structures of interest.

4. A method as claimed in claim 1 comprising obtaining the first volume data set as a number of successive computed tomographic slice images, with image data of each slice image described with Cartesian coordinates and comprising, for segmenting the imaged body surface:
implementing a coordinate transformation for each slice image to polar coordinates with regard to a straight line (G) that proceeds through the imaged subject and that is aligned substantially at a right angle to the individual slice images;
determining contours that are imaged in each transformed slice image and that are associated with the imaged surface;
transforming the image points of the determined contours back into the coordinate system associated with the first volume data set; and
re-extracting image points along the contours for representing the surface of the imaged first subject transformed in the plane.

5. A method as claimed in claim 4 comprising producing a fourth volume data set in which the image points of the third volume data set are transformed back into the coordinate system associated with the first volume data set.

6. A method as claimed in claim 5 comprising representing an image associated with the fourth volume data set by volume rendering.
